(19) United States
(12) Patent Application Publication
(10) Pub. No.: US 2015/0230983 A1
(43) Pub. Date: Aug. 20, 2015

(54) SYSTEMS AND METHODS FOR AN ELECTROCAPILLARY PUMP FOR AN INTRAOCULAR IMPLANT

(71) Applicant: NOVARTIS AG, Basel (CH)

(72) Inventor: ANDREW D. JOHNSON, Tustin, CA (US)

(73) Assignee: NOVARTIS AG, Basel (CH)

(21) Appl. No.: 14/181,840

(22) Filed: Feb. 17, 2014

(51) Int. Cl. A61F 9/007

(52) U.S. Cl. CPC A61F 9/00781

(57) ABSTRACT

A microfluidic pump for implantation proximate an eye of a patient is disclosed herein. The microfluidic pump includes a first microfluidic actuator and a second microfluidic actuator, each with first and second chambers coupled by a channel. An electrode is in each of the first and second chambers, and the electrodes are activated to displace the first slug positioned within the channels. A flow path of the pump includes a plurality of reservoirs, one of the reservoirs being aligned with each of the first, second, third, and fourth chambers. Additionally, a flexible membrane is disposed between the flow path and the first and second microfluidic actuators. The membrane is manipulated in a manner and frequency that results in the movement of flow through the flow path.

### BACKGROUND

The present disclosure relates generally to microfluidic pump systems and methods for ophthalmic treatments. More particularly, the present disclosure relates to microfluidic pump systems that may be used to drain fluid from an eye having a potentially harmful excess thereof.

Glaucoma, a group of eye diseases affecting the retina and optic nerve, is one of the leading causes of blindness worldwide. Most forms of glaucoma result when the intraocular pressure (IOP) increases to pressures above normal for prolonged periods of time. IOP can increase due to high resistance to the drainage of the aqueous humor. Left untreated, an elevated IOP causes irreversible damage to the optic nerve and retinal fibers resulting in a progressive, permanent loss of vision.

The eye's ciliary body continuously produces aqueous humor, the clear fluid that fills the anterior segment of the eye (the space between the cornea and lens). The aqueous humor flows out of the anterior chamber (the space between the cornea and iris) through the trabecular meshwork and the uveoscleral pathways, both of which contribute to the aqueous humor drainage system. The delicate balance between the production and drainage of aqueous humor determines the eye's IOP.

FIG. 1 is a diagram of the front portion of an eye 100 that helps to explain the processes of glaucoma. In FIG. 1, representations of the lens 110, cornea 120, iris 130, ciliary body 140, trabecular meshwork 150, Schlemm's canal 160, and the edges of the sclera 170 are pictured. Anatomically, the anterior segment of the eye includes the structures that cause elevated IOP which may lead to glaucoma. Aqueous humor fluid is produced by the ciliary body 140 that lies beneath the iris 130 and adjacent to the lens 110 in the anterior segment of the eye. This aqueous humor washes over the lens 110 and iris 130 and flows to the drainage system located in the angle of the anterior chamber 180. The edge of the anterior chamber, which extends circumferentially around the eye, contains structures that allow the aqueous humor to drain. The trabecular meshwork 150 is commonly implicated in glaucoma. The trabecular meshwork 150 extends circumferentially around the anterior chamber. The trabecular meshwork 150 generates resistance to the outflow of aqueous humor and provides a back pressure that directly relates to IOP. Schlemm's canal 160 is located beyond the trabecular meshwork 150. Schlemm's canal 160 is fluidically coupled to collector channels (not shown) allowing aqueous humor to flow out of the anterior chamber 180. The sclera 170, the white of the eye, connects to the cornea 120, forming the outer, structural layer of the eye. The two arrows in the anterior segment of FIG. 1 show the flow of aqueous humor from the ciliary bodies 140, over the lens 110, over the iris 130, through the trabecular meshwork 150, and into Schlemm's canal 160 and its collector channels.

As part of a method for treating glaucoma, a doctor may implant a device in a patient's eye. The device may monitor the pressure in a patient's eye and facilitate control of that pressure by allowing excess aqueous humor to flow from the anterior chamber of the eye to a drainage site, relieving pressure in the eye and thus lowering IOP. Under certain conditions, the drainage site may become obstructed or pressurized. In such circumstances, the obstruction of the drainage site may lead to an undesired cessation of draining, causing the pressure to rise to a potentially harmful level within the anterior chamber of the eye.

The system and methods disclosed herein overcome one or more of the deficiencies of the prior art.

### SUMMARY

In one exemplary aspect, the present disclosure is directed to a microfluidic pump for implantation proximate an eye of a patient. The microfluidic pump includes a first microfluidic actuator that has a first chamber and a second chamber coupled by a first channel. An electrode is in each of the first and second chambers and the electrodes are activated to displace a first slug positioned within the first channel. The microfluidic pump also includes a second microfluidic actuator that has a third chamber and a fourth chamber coupled by a second channel, with an electrode in each of the third and fourth chambers to displace a second slug positioned within the second channel. A flow path of the microfluidic pump includes a plurality of reservoirs, one of the reservoirs being aligned with each of the first, second, third, and fourth chambers. Additionally, a flexible membrane is disposed between the flow path and the first and second microfluidic actuators.

In another exemplary aspect, the present disclosure is directed to an intraocular device for implantation proximate an eye of a patient. The intraocular device includes a plate sized for positioning next to the globe of the eye; a first drainage tube having a proximal end and a distal end, the distal end configured for insertion into the eye, and a microfluidic pump. The microfluidic pump is disposed within the plate and coupled to the proximal end of the first drainage tube. The microfluidic pump includes a first microfluidic actuator and a second microfluidic actuator and a flow path that has a plurality of reservoirs. Each of the first and second microfluidic actuators has a first chamber and a second chamber coupled by a first channel, with an electrode in each of the first and second chambers to displace a first slug positioned within the first channel. In the flow path, one of the reservoirs is aligned with each of the first chambers and the second chambers of the first and second microfluidic actuators. Additionally, the intraocular device includes a flexible membrane disposed between the flow path and the first and second microfluidic actuators of the microfluidic pump.

In yet another exemplary aspect, the present disclosure is directed to a method of achieving a desired intraocular pressure in an eye of a patient. The method includes steps of coupling an inlet of a microfluidic pump to an anterior chamber of the eye and of applying an electric potential to electrodes of a first microfluidic actuator to induce a surface tension gradient along a first slug within an electrolytic fluid. The application of the electric potential causes the slug to move in a first direction within a channel. The method further includes a step of applying an electric potential to electrodes of a second microfluidic actuator to induce a surface tension gradient along a second slug within the electrolytic fluid to force a fluid through a flow path of the microfluidic pump.

It is to be understood that both the foregoing general description and the following drawings and detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following.

### BRIEF DESCRIPTION OF THE DRAWINGS

The accompanying drawings illustrate embodiments of the devices and methods disclosed herein and together with the description, serve to explain the principles of the present disclosure.

FIG. 1 is a cross-sectional view of the front portion of an eye.

FIG. 2 is a perspective view of an intraocular implant device that carries a microfluidic chamber.

FIG. 3 is a perspective view of an intraocular implant device as situated proximate an eye according to an exemplary aspect of the present disclosure.

FIG. 4 is a cross-sectional view of a microfluidic pump actuator such as may be used in the intraocular implant according to exemplary aspects of the present disclosure.

FIG. 5A is a top view of a lower portion of a microfluidic pump according to exemplary aspects of the present disclosure.

FIG. 5B is a top view of an upper portion of a microfluidic pump according to exemplary aspects of the present disclosure.

FIGS. 6A, 6B, 6C, and 6D are cross-sectional views of the microfluidic pump of FIGS. 5A and 5B, taken along a flow path therein, in various stages of operation according to exemplary aspects of the disclosure.

FIG. 7 is a flowchart showing a method of maintaining a desired intraocular pressure in an eye of a patient according to exemplary aspects of the present disclosure.

FIG. 8 is a flowchart showing a method of maintaining a desired intraocular pressure in an eye of a patient according to exemplary aspects of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

The present disclosure relates generally to systems and methods for maintaining a desired intraocular pressure in an eye of a patient by using an intraocular device that contains a bi-directional, microfluidic pump. In some aspects described herein, the microfluidic pump includes two or more microfluidic actuators coupled to a flow path that drains fluid from the anterior chamber 180 of the eye 100. Because of its arrangement, the microfluidic pump can drain fluid even when a pressure is higher at a drainage site than within the anterior chamber 180 or there is added resistance preventing the desired drainage. The systems and methods disclosed herein may enable better control and maintenance of intraocular pressure, potentially providing more effective treatment and greater customer satisfaction. In some aspects, the intraocular device is an intraocular pressure (IOP) controlling device, such as a glaucoma drainage device (GDD) that alleviates elevated IOP in a patient's eye.

FIG. 2 is a schematic diagram of an exemplary intraocular implant or device 200 useable in the monitoring and treatment of a patient's eye. As depicted, the intraocular device 200 is a GDD. The intraocular device 200 includes a body referred to herein as a plate 210 with a first drainage tube 220 that extends from the plate 210. The first drainage tube 220 includes a proximal end portion 222 that couples the tube to one or more structures internal to the plate 210, such as a microfluidic pump as will be described herein. A distal end portion 224 of the first drainage tube 220 may be coupled to the eye of a patient to allow for the monitoring of pressure and/or the drainage of fluid. Embodiments of the intraocular device 200 may include additional tubes for priming and/or for the detection of pressure at other location. As illustrated, the intraocular device 200 also includes a second drainage tube 230 that has a distal end 232. The second drainage tube 230 may be connected to the other end of the microfluidic pump. Thus, the first drainage tube 220 and the second drainage tube 230 form part of a microfluidic pump system. An associated microfluidic pump system will be discussed in greater detail below.

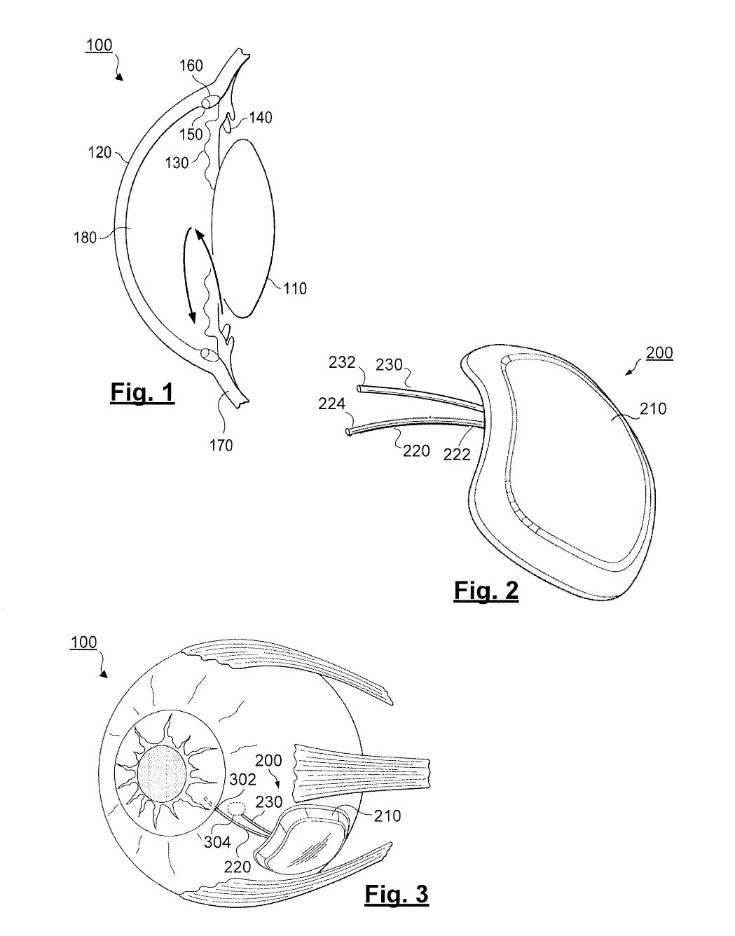

FIG. 3 is a schematic diagram of an eye 100 (the anterior portion of which is shown in cross-section in FIG. 1) of a patient whose IOP is being monitored and/or who is receiving treatment with the intraocular device 200. The intraocular device 200 may be a GDD as depicted in FIG. 2. The plate 210 may include or be arranged to carry various components of an IOP control system, including for example, one or more of a power source, a processor, a memory, a data transmission module, and a flow control mechanism (e.g., a valve system). It may also carry one or more pressure sensor systems, including one or more pressure sensors, to monitor the pressures in and around the eye, including an intraocular pressure. This pressure may be used by other systems within the intraocular device 200, such as drainage systems having microfluidic pumps that are used to regulate the intraocular pressure, often by lowering the pressure. Thus, the plate 210 may also include a pump system.

The plate 210 is configured to fit at least partially within the subconjunctival space and is sized within a range between about 15 mm×12 mm to about 30 mm×15 mm and has a thickness less than about 2 mm thick, preferably less than about 1 mm thick. The plate 210 may be formed to the radius of the eye globe (about 0.5 inches). It may be rigid and preformed with a curvature suitable to substantially conform to the globe or it may be flexible and can flex to conform to the globe. Some embodiments are small enough that conforming to the globe provides little benefit in comfort or implantation technique. The above dimensions are exemplary only, and other sizes and arrangements are contemplated herein.

In some embodiments, the first drainage tube 220 extends from an anterior side of the plate 210 and is sized and arranged to extend into the anterior chamber 180 (as seen in FIG. 3) of the eye through a surgically formed opening 302 in the sclera. The first drainage tube 220 is used to facilitating drainage and may also be used to measure the pressure within the anterior chamber 180. The first drainage tube 220 includes a first open end that may be disposed at a location where pressure measurements may be desired (in this instance within the anterior chamber 180) and from which fluid is drained, and at least one lumen that extends to a second open end that is disposed within or connected to the plate 210. Prior to placement around a patient's eye as depicted in FIG. 3, a chamber within the plate 210 may be primed by the injection of liquid that displaces a gas from the chambers, channels, and/or valves within the device 200. The liquid may be injected through the tube 220 until some liquid may exit through an outlet. In some instances, the outlet is provided by the second drainage tube 230, which may facilitate drainage at a removed location. As illustrated in FIG. 3, the distal end 232 of the second drainage tube 230 is positioned under a bleb 304 formed on the exterior of the sclera 170. The bleb 304 may protect and maintain the distal end 232 of the drainage tube 230 in a desired location to facilitate drainage and/or measure atmospheric pressure experienced by the eye 100. In some embodiments, the fluid entering the device 200 through the first drainage tube 220 may be drained, not through the second drainage tube 230, but through a drain integrated in the plate 210, such as on an upper surface. In such embodiments, the second drainage tube 230 may be absent.

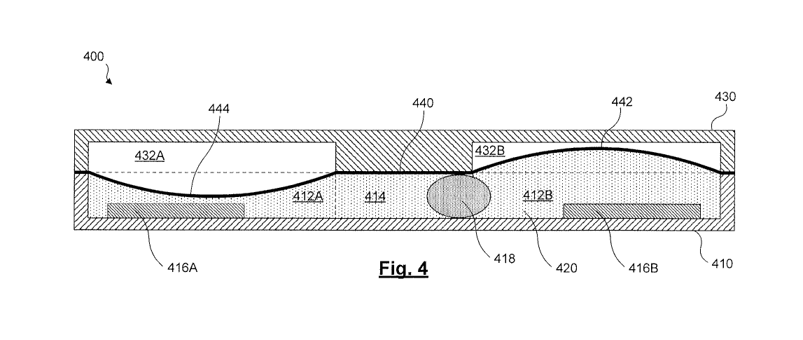

FIG. 4 is a cross-sectional view of an exemplary microfluidic pump actuator 400, such as may be configured within the intraocular device 200 as seen in FIGS. 2 and 3. The actuator 400 includes an actuating portion 410 and an actuated portion 430. In some embodiments, the actuating portion 410 is provided by a first substrate or first portion of a substrate, and the actuated portion 430 is provided by a second substrate or second portion of the substrate. The substrates may be made from glass, silicon, silicone, or a biocompatible polymer such as Parylene or polyimide, and may be milled, molded, or etched to provide their desired forms. FIG. 4 illustrates a flexible membrane 440 disposed in between the actuating portion 410 and the actuated portion 430. In some embodiments, multiple flexible membranes are provided that each cover limited portions of the actuating portion 410 and the actuated portion 430.

The actuating portion 410 includes a first chamber 412A coupled to a second chamber 412B by a narrow channel 414. In this embodiment, the narrow channel 414 is formed by the actuating portion 410 on the bottom and by the actuated portion 430 on top. The chambers 412A and 412B are defined by the internal walls of the substrate that forms the actuating portion 410 on the sides and on the bottom and by the flexible membrane 440 on top. As used herein, terms such as “bottom”, “top”, and “sides”, are used to describe the relationships of features and are used with reference to the particular orientation of aspects as illustrated in the figures; the terms do not prescribe any particular orientation. For example, in some embodiments of the pump actuator 400, the actuating portion 410 is above the actuated portion 430.

Within each of the chambers 412A and 412B is an electrode 416A and 416B, respectively. The electrodes 416A and 416B may be formed from platinum, gold, or another conductive material. Preferably, the conductive material of the electrodes 416A and 416B is a biocompatible conductive material. Within the channel 414 is a conductive, immiscible slug 418. The slug 418 is surrounded by an electrolytic solution 420, such as a salt solution, that fills the chambers 412A and 412B and the remainder of the channel 414. In the illustrated embodiment, the slug 418 is formed from gallium. In other embodiments, the slug 418 may be formed from mercury or another conductor that is liquid at the temperature of the human body. When the actuator 400 is not activated, the slug 418 is positioned within the center of channel 414, such that it is halfway between the electrodes 416A and 416B. When activated by an electric potential applied to the electrodes 416A and 416B, a gradient is formed in the surface tension along the slug 418 and immersed in the electrolytic solution 420. The gradient in surface tension produces a force that causes the slug 418 to move within the channel 414 toward either the electrode 416A or the electrode 4168 depending on whether the electric potential is positive or negative. The gradient in surface tension γ is related to the electrical potential U by equation (1).

γ=γ₀−½C(U−U₀) (1)

In equation (1), C is the capacitance per unit area of the electrical double layer than forms between the slug 418 and the electrolytic solution 420.

As illustrated in FIG. 4, the slug 418 is at an extreme end of the channel 414 due to an applied electric potential. As the slug 418 moves toward the chamber 412B the slug increases the pressure within the chamber 412B, which results in the deflection 442 of the membrane 440 away from the chamber 412B. The deflection 442 effectively expands the volume of the chamber 412B. A corresponding deflection 444 occurs in the portion of the membrane 440 that partially defines the chamber 412A. The deflection 444 is toward the chamber 412A, such that the volume thereof is decreased. If the electric potential is removed, the differences in pressure between the chambers 412A and 412B may naturally adjust, forcing the slug 418 back toward the middle of the channel 414. A negative electric potential applied to the electrodes 416A and 416B causes the slug 418 to move to the other extreme of the channel 414, which in turn causes the portion of membrane 440 over the chamber 412A to deflect away from the chamber 412A and the portion of the membrane 440 over the chamber 412B to deflect into the chamber 412B.

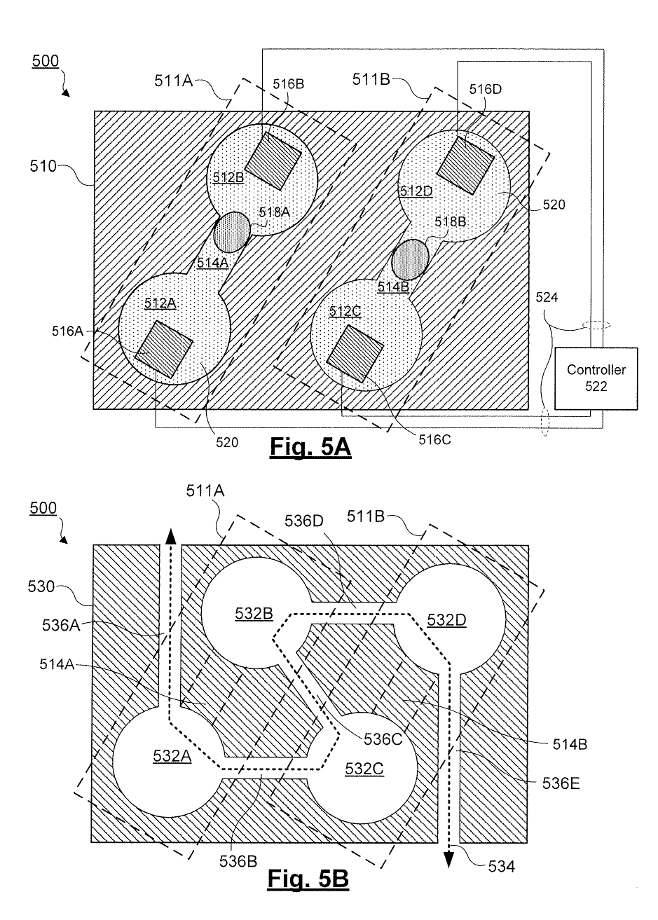

As the portions of the membrane 440 over the chambers 412A and 412B deflect away from and into the respective chambers, enlarged areas or reservoirs in a flow path provided in the actuated portion 430 are significantly affected. As illustrated in FIG. 4, the actuated portion 430 includes two enlarged areas or reservoirs 432A and 432B. The volume of the reservoir 432A increases as the portion of the membrane 440 deflects into the chamber 412A and decreases as it deflects away from the chamber 412A, drawing fluid in and pushing it out along the flow path. Similarly, the volume of the reservoir 432B increases as the portion of the membrane 440 deflects into the chamber 412B and decreases as it deflects away from the chamber 412B. More detail on the flow path, of which part is shown by the reservoirs 432A and 432B, is provided in FIGS. 5A and 5B.

Referring now to FIGS. 5A and 5B, top views of a microfluidic pump system 500 that is suitable for implantation next to the eye of a patient are shown. FIG. 5A illustrates an actuating portion 510 of the microfluidic pump system 500. The actuating portion 510 includes two actuators like the actuator 400 of FIG. 4. Other embodiments of the actuator portion 510 may include more actuators, but as illustrated, the actuating portion 510 of the pump system 500 includes a first actuator 511A and a second actuator 511B. Each of the actuators 511A and 511B includes two chambers coupled together by a channel, which are filled with an electrolytic solution, with a slug filling a circular cross-section of the channel. Thus the actuator 511A includes a chamber 512A coupled by a channel 514A to a chamber 512B. A slug 518A is disposed within the channel 514A and is moveable to either extreme within the channel 514A by the application of an electric potential on the electrodes 516A and 516B. The slug 518A fills the channel 514A so that the electrolytic solution 520 does not appreciably move past the slug 518A from one chamber to the other. Similarly, the actuator 511B includes chambers 512C and 512D coupled together by a channel 514B within a slug 518B that is moved by application of electric potential to the electrodes 516C and 516D. A membrane 540 (not shown in FIGS. 5A and 5B) is positioned over the actuating portion 510 such that it retains the electrolytic solution 520 within the chambers 512A-D and channels 514A-B. In some embodiments, each of chambers 512A-D has a separate membrane.

As illustrated, an electric potential is applied to the actuator 511A such that the slug 518A is nearer to the electrode 516B. Accordingly, the membrane 540 over the chamber 512B is deflected away from the chamber 512B by an amount that corresponds to the membrane 540 over the chamber 512A being deflected into the chamber 512A. Also as illustrated, the actuator 511B is in an unactivated or dormant state. In this state, the slug 518B is in the center or middle of the channel 514B and the membrane 440 is generally flush with a top surface of the actuating portion 510, such that the membrane 540 over the chambers 512C and 512D is not deflected into or away from either chamber 512C or 512D. A controller 522 is coupled by a plurality of wires or leads 524 to each of the electrodes 516A-D to allow for the selective application of electric potential to move the slugs 518A and 518B to deflect the membrane 540. The controller 522 may be coupled to receive a signal, based on an intraocular pressure, to stop or start a drainage process using the pump system 500.

FIG. 5B illustrates a top view of the actuated portion 530 of the pump system 500. As illustrated, the actuated portion includes a plurality of reservoirs, one reservoir for each chamber in the actuating portion 510. The enlarged areas or reservoirs are aligned with the underlying chambers, such that the reservoir 532A is aligned with chamber 512A, reservoir 532B is aligned with chamber 512B, reservoir 532C is aligned with chamber 512C, and reservoir 532D is aligned with chamber 512D. While the chambers 512A and 512B are not fluidically coupled with the chambers 512C and 512D, each of the reservoirs 532A-D are part of a flow path 534 that connects the reservoirs 532A-D by a series of channels, including channels 536A, 536B, 536C, 536D, 536E as illustrated in FIG. 5B. In some embodiments, the controller 522 is configured to pump fluid, such as aqueous humor, through the flow path 534 from the channel 536A out through the channel 536E. Some embodiments of the pump system 500 include one or more check valves within the flow path 534. For example, the flow path 534 may include a check valve within channel 536A to prevent back flow through the pump system 500.

However, as illustrated, in FIGS. 5A and 5B, the pump system 500 is configured to pump aqueous humor in either direction. Thus, while the channel 536A may be referred to as an inlet channel and the channel 536E may be referred to as an outlet channel, channels 536A and 536E may both serve as an inlet or outlet depending on the flow direction provided by the controller 522. The reservoirs 532A-D have significantly larger volumes than the channels 536A-E, allowing the deflections of the membrane 540 within the chambers 512A-D and reservoirs 532A-D to cause greater fluid displacement in the channels 536A-E.

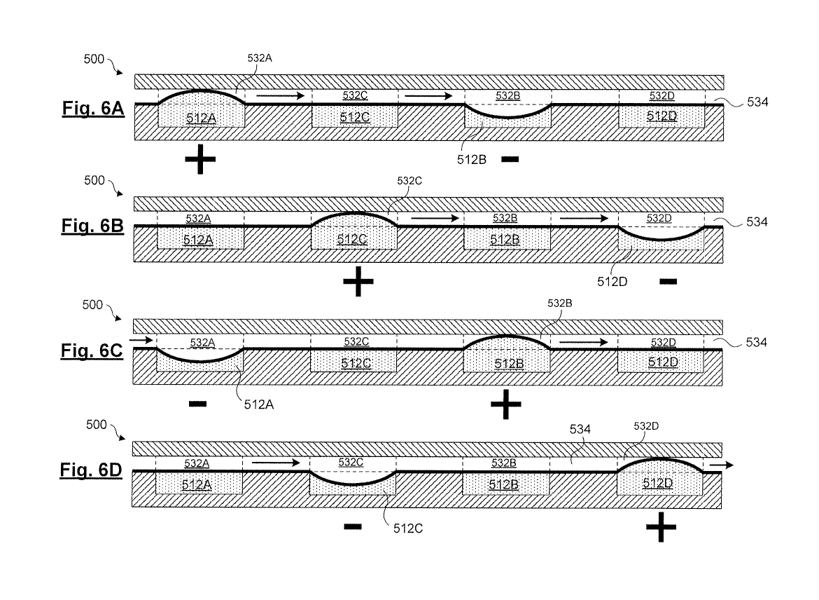

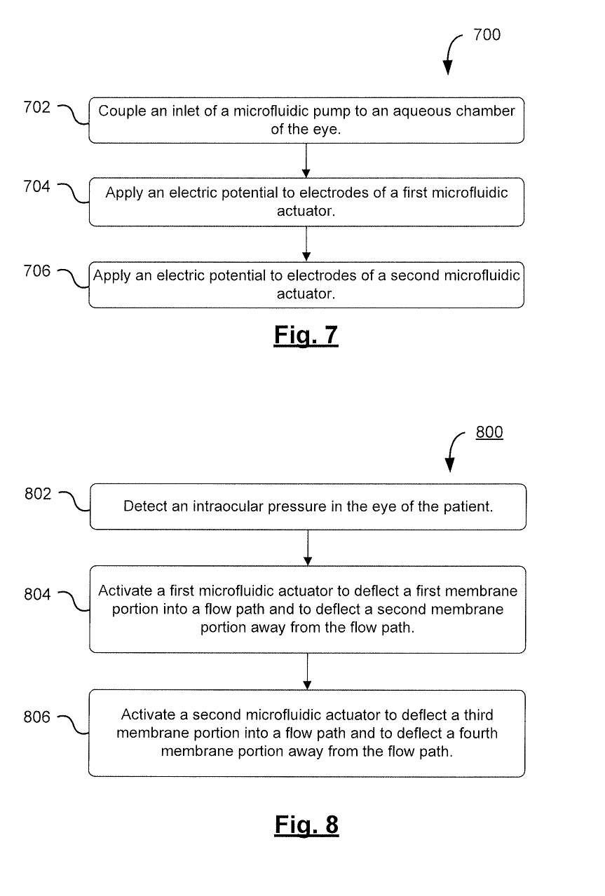

Referring now to FIGS. 6A-D, shown therein is a series of cross-sectional views of the chambers 512A-D and the channels 536A-E as seen along the flow path 534. The cross-sectional views in each of FIGS. 6A-D are not actual cross-sections as could be obtained from cross-sectioning the system pump as illustrated in FIGS. 5A and 5B, but are composite illustrations that more clearly depict an embodiment of how the pump system 500 functions under the control of the controller 522 along the flow path 534.

As shown in FIG. 6A, an electric potential is applied to the electrodes 516A and 516B in chambers 512A and 512B of actuator 511A. The electric potential causes the slug 518A to move within the channel 514A toward the electrode 516A. The movement of the slug 518A causes the chamber 512A to expand, deflecting the membrane 540 into the reservoir 532A, which is filled with aqueous humor. At the same time, and to the same extent, as the membrane 540 deflects into the reservoir 532A, it deflects into the chamber 512B, away from the reservoir 532B. The combined movements of the membrane 540, as electrolytic fluid 520 enters the chamber 512A and leaves the chamber 512B causes the aqueous humor in the flow path between reservoirs 532A and 532B to move toward the reservoir 512D.

In FIG. 6B, an electric potential is applied to the electrodes 516C and 516D in the actuator 511B of the pump system 500. The slug 518B moves towards the electrode 516C, pushing fluid into the chamber 512C and out of the chamber 512D. This deflects the membrane 540 into the reservoir 532C pushing out the fluid. It also deflects the membrane 540 into the chamber 512D, pulling fluid into the reservoir 532D, thereby moving the aqueous humor between the reservoirs 532C and 532D along the flow path 534 to the channel 536E.

In FIG. 6C, the negative of the electric potential applied in FIG. 6A is applied to the electrodes 516A and 516B in the chambers 512A and 512B. The slug 518A moves toward the chamber 512B, deflecting the membrane 540 over the chamber 512B into the reservoir 532B, pushing fluid out of the reservoir 532B. At the same time, the reservoir 532A expands pulling fluid in.

And in FIG. 6D, the negative of the electric potential applied in FIG. 6B is applied to the electrode 516C and 516D in the chambers 512C and 512D of the actuator 511B. The changes to the reservoirs 532C and 532D push aqueous humor through the flow path 534.

The activations, both positive and negative, and the dormant periods as illustrated in FIGS. 6A-D pull aqueous humor into the pump system 500 through the channel 536A and out the channel 536E. In order to achieve a desired flow rate, the sequence illustrated in FIGS. 6A-D is repeated continuously at corresponding frequency, such that the activation illustrated in FIG. 6A follows the activation illustrated in FIG. 6D. A high cycling frequency results in a higher flow rate. In some embodiments, one activation is maintained while another is performed. For example, the electric potential as seen applied in FIG. 6A may be held or maintained while the electric potential as seen in FIG. 6B is applied. Thus, the membrane 540 may be deflected into the reservoir 532C while the membrane is already asserted in a deflected state into the reservoir 532A.

Additionally, the activations illustrated in FIGS. 6A-D may be performed in the opposite sequence in order to pump fluid out of the channel 536A. Thus, the pump system 500 as illustrated in FIGS. 5A-B and 6A-D is a bi-directional pump. The pump system 500 may be used to drain aqueous humor from the anterior chamber 180 of the eye 100 of FIG. 1 even when the bleb 304 of FIG. 3 provides resistance to such drainage. Under certain conditions, the bleb 304 of FIG. 3 may become pressurized and may cause a higher pressure than a potential harmful pressure within the anterior chamber of the eye. The pump system 500 may provide sufficient capacity to overcome the pressure of the bleb 304, thereby draining excess aqueous humor from the eye even in the presence of resistance generated by the bleb 304.

Because the flow path 534 does not contact the electrolytic solution 520 and does not contact the slug 518, these liquids are not depleted overtime. Additionally, no chemical reactions are required to actuate the pump system 500, thereby avoiding unwanted by-products.

FIG. 7 shows a method 700 of achieving a desired intraocular pressure in an eye of a patient according to exemplary aspects of the present disclosure. As illustrated, the method 700 includes several enumerated steps. Embodiments, of the method 700 may include additional steps before, after, in between, and/or as part of the enumerated steps. Thus, an embodiment of the method 700 begins in step 702. In which a surgeon couples an inlet of a microfluidic pump to an anterior chamber of the eye. For example, a surgeon may insert a drainage tube 220 of a device 200 (as seen in FIGS. 2 and 3) through an incision 302 (as seen in FIG. 3) into the anterior chamber 180 of the eye 100 (as seen in FIG. 1). The intraocular device 200 includes a microfluidic pump system 500 (as seen in FIGS. 5A-B and 6A-D).

The surgeon may then couple an outlet of the microfluidic pump to a drainage site formed on the eye. This drainage site may develop a bleb. For example, the surgeon may couple the drainage tube 230 to the 304 (as seen in FIG. 3). In step 704, an electric potential is applied to electrodes of a first microfluidic actuator, causing a slug within a channel of the first microfluidic actuator to move from a central position to an extreme position within the channel. For example, the electric potential illustrated as applied in FIG. 6A is applied by a controller 522 of FIG. 5. The electric potential applied to electrodes 516A and 516B causes the slug 518A to move within the channel 514A toward the electrode 516B. In step 706, an electric potential is applied to electrodes of a second microfluidic actuator, thereby forcing a fluid within a flow path of the microfluidic pump out of the microfluidic pump. For example, the electric potential as depicted as applied in FIG. 6B is applied to the electrodes 516C and 516D of the actuator 511B. This forces the aqueous humor in the flow path 534 to continue moving out of the outlet of the pump system 500.

In some embodiments, the steps of the method 700 may be performed such that aqueous humor flows through the flow path 534 from the channel 536A through channel 536E or such that is flow from the channel 536E through channel 536A. By cycling the application of electric potential to the electrodes as shown in FIGS. 6A-D at an appropriate frequency, the method 700 may provide a desired flow rate. Thus, step 704 may be repeated after the performance of step 706.

Referring now to FIG. 8, a method 800 of maintaining a desired intraocular pressure in an eye of a patient is illustrated according to exemplary aspects of the present disclosure. As illustrated, the method 800 includes several enumerated steps. However, embodiments of the method 800 may also include additional steps before, after, in between, and/or as part of the enumerated steps. As illustrated, the method 800 begins in step 802 in which an intraocular pressure is detected in the eye of the patient. This may be done by a pressure sensor provided in the device 200 as seen in FIGS. 2 and 3. In step 804, a first microfluidic actuator is activated to deflect a first membrane portion into a flow path and to deflect a second membrane portion away from the flow path. When so activated, a fluid in the flow path moves from the first membrane portion toward the second membrane portion. For example, the microfluidic actuator 511A, of FIGS. 5A-B and 6A-D, may have an electric potential applied to the electrodes 516A and 516B thereof, which deflects a portion of the membrane 540 over the chamber 512A up into the reservoir 532A. The electric potential also deflections a portion of the membrane 540 over the chamber 512B toward the chamber 512A, thereby increasing the volume of the reservoir 532B. This causes aqueous humor in the flow path 534 to flow from the reservoir 532A through the reservoir 532C to the reservoir 532B. In step 806, a second microfluidic actuator is activated to deflect a third membrane portion into a flow path and to deflect a fourth membrane portion away from the flow path, such that the fluid in the flow path moves from the third membrane portion toward the fourth membrane portion. For example, the microfluidic actuator 511B may have an electric potential applied to it as seen in FIG. 6B.

Additional embodiments may include the other activations as illustrated in FIGS. 6C and 6D. The fluid may be directed in either direction as part of the method 800. The activations are cycled continuously at specific frequency to provide a desired flow rate. In some embodiments, the intraocular pressure in the eye of the patient may be monitored continuously. If, after some number of cycles of the pump, the pressure sensor provided in the device indicates the intraocular pressure has decreased below an appropriate range, the cycling of activations may be automatically stopped.

The systems and methods disclosed herein may be used to provide better performance for intraocular devices, such as increased control over drainage from the anterior chamber to regulate the IOP. This may be done by using microfluidic actuators in a bi-directional pump. This may result in more effective treatment and more accurate data, thereby improving the overall clinical result.

Persons of ordinary skill in the art will appreciate that the embodiments encompassed by the present disclosure are not limited to the particular exemplary embodiments described above. In that regard, although illustrative embodiments have been shown and described, a wide range of modification, change, combination, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A microfluidic pump for implantation proximate an eye of a patient, the microfluidic pump comprising:
a first microfluidic actuator having a first chamber and a second chamber coupled by a first channel, with an electrode in each of the first and second chambers to displace a first slug positioned within the first channel;
a second microfluidic actuator having a third chamber and a fourth chamber coupled by a second channel, with an electrode in each of the third and fourth chambers to displace a second slug positioned within the second channel;
a flow path comprising a plurality of reservoirs, one of the reservoirs being aligned with each of the first, second, third, and fourth chambers; and
a flexible membrane disposed between the flow path and the first and second microfluidic actuators.

2. The microfluidic pump of claim 1, wherein the first and second microfluidic actuators are provided in a first substrate and the flow path is provided in a second substrate.

3. The microfluidic pump of claim 2, wherein the first substrate is a glass substrate.

4. The microfluidic pump of claim 1, wherein the flexible membrane comprises:
a first membrane portion between the first chamber and a first reservoir of the flow path;
a second membrane portion between the second chamber and a second reservoir of the flow path;
a third membrane portion between the third chamber and a third enlarged portion area of the flow path; and
a fourth membrane portion between the fourth chamber and a fourth reservoir of the flow path.

5. The microfluidic pump of claim 1, wherein the flexible membrane is positioned between the first chamber and a first reservoir and another flexible membrane is positioned between the second chamber and a second reservoir.

6. The microfluidic pump of claim 1, wherein the electrodes in the first, second, third, and fourth chambers are activated by a controller in sequence to force a liquid through the flow path from the inlet channel to the outlet channel.

7. The microfluidic pump of claim 6, wherein the electrodes in the first, second, third, and fourth chambers are activated by the controller in a second sequence to force the liquid through the flow path from the outlet channel to the inlet channel.

8. The microfluidic pump of claim 6, wherein a flow rate of the microfluidic pump depends on a frequency of the first sequence.

9. The microfluidic pump of claim 1, wherein further comprising a first tube coupling the inlet channel to an anterior chamber of the eye.

10. The microfluidic pump of claim 1, further comprising a second tube coupling the outlet channel to a bleb formed on in the eye.

11. The microfluidic pump of claim 1, wherein when an electric potential is applied to the electrodes in the first and second chambers, the slug moves toward the first chamber and when a reverse electric potential is applied to the electrodes in the first and second chambers the slug moves toward the second chamber.

12. The microfluidic pump of claim 1, wherein the channel has a circular cross-section.

13. The microfluidic pump of claim 12, wherein half of the channel is formed from a first substrate that includes the first and second microfluidic pump actuators and half of the channel is formed from a second substrate that includes the flow path.

14. An intraocular device for implantation proximate an eye of a patient, the intraocular device comprising:
a plate sized for positioning next to the eye;
a first drainage tube having a proximal end and a distal end, the distal end configured for insertion into the eye;
a microfluidic pump disposed within the plate and coupled to the proximal end of the first drainage tube, the microfluidic pump comprising:
a first microfluidic actuator and a second microfluidic actuator, each of the first and second microfluidic actuators having a first chamber and a second chamber coupled by a first channel, with an electrode in each of the first and second chambers to displace a first slug positioned within the first channel;
a flow path comprising a plurality of reservoirs, one of the reservoirs being aligned with each of the first chambers and the second chambers of the first and second microfluidic actuators; and
a flexible membrane disposed between the flow path and the first and second microfluidic actuators.

15. The intraocular device of claim 14, wherein the flexible membrane comprises:
a first membrane portion between the first chamber of the first microfluidic actuator and a first reservoir of the flow path; and
a second membrane portion between the second chamber of the first microfluidic actuator and a second reservoir of the flow path.

16. The intraocular device of claim 14, further comprising a second drainage tube, wherein the microfluidic pump is configured to pump fluid between the first drainage tube and the second drainage tube.

17. A method of achieving a desired intraocular pressure in an eye of a patient, the method comprising:
coupling an inlet of a microfluidic pump to an anterior chamber of the eye;
applying an electric potential to electrodes of a first microfluidic actuator to induce a surface tension gradient along a first slug in an electrolytic fluid, causing the slug to move in a first direction within a channel; and
applying an electric potential to electrodes of a second microfluidic actuator to induce a surface tension gradient along a second slug in another electrolytic fluid to force a fluid within a flow path of the microfluidic pump out of the microfluidic pump.

18. The method of claim 17, wherein the fluid is forced out of the microfluidic pump from an outlet to a pressurized or obstructed drainage site.

19. The method of claim 17, wherein fluid is pumped through a flow path as the electric potential is applied to the electrodes of the first microfluidic actuator and to the electrodes of the second microfluidic actuator.

20. The method of claim 19, wherein the flow path comprises a plurality of channels and a plurality of reservoirs, the reservoirs being connected in sequence by the channels.
